(19) United States
(12) Patent Application Publication
(10) Pub. No.: US 2003/0012828 A1
(43) Pub. Date: Jan. 16, 2003

(54) Topical medicine and process and apparatus for making same

(76) Inventor: Joe Ajrulahi, Garden City Beach, SC (US)

Correspondence Address: B. Craig Killough, Barnwell Whaley Patterson & Helms, LLC, Suite 300, 134 Meeting Street, Charleston, SC, 29401, US

(21) Appl. No.: 09/905,770

(22) Filed: Jul. 13, 2001

(51) Int. Cl. A61K 35/78

(52) U.S. Cl. 424/725

(57) ABSTRACT

A medicine that may be topically applied for treatment of arthritis, dermatological problems, such as abnormal pigmentation. The medicine may comprise vegetables or fruit, including extracts of fruit or vegetable juices that have undergone a fermentation process. These ingredients are combined and processed by a device that applies a current through a metallic electrode, which contributes metal to the medicine. The topical medicine so produced may comprise jalapeno pepper and copper, and the resulting medicine is an improvement upon other pepper-based products such as capsaicin. In another process, the device may be used to remove alloys from metals. Alloys may be removed from gold, such as gold jewelry, and from other metals such as copper.

### FIELD OF THE INVENTION

This invention relates to a topical medicine preparation. A process for making the topical medicine preparation, and an apparatus for practicing the process are also presented.

### SUMMARY OF THE INVENTION

The present invention is a medicine which may be topically applied for treatment of arthritis, dermatological problems, such as abnormal pigmentation. The topical medicine so produced comprises jalapeno pepper and copper. The medicine may comprise other vegetables, and fruit and/or vegetable juices, including fruit or vegetable juices that have undergone a fermentation process. These ingredients are combined and processed according to the present invention. A juice is prepared, and is processed through the apparatus described herein to produce the medicine for topical application. The resulting medicine is an improvement upon other pepper-based products such as capsaicin.

In another process, the device disclosed herein may be used to remove alloys from metals. Alloys may be removed from gold, such as gold jewelry, and from other metals such as copper.

### DESCRIPTION OF THE DRAWINGS

FIG. 1 shows a diagram of the apparatus that is used to prepare the topical medicine according to the method of the present invention, with juice from a plant present therein.

FIG. 2 shows a diagram of the apparatus that is used to prepare the topical medicine according to the method of the present invention, with the powder from which the topical medicine is prepared present below a supernatant liquid.

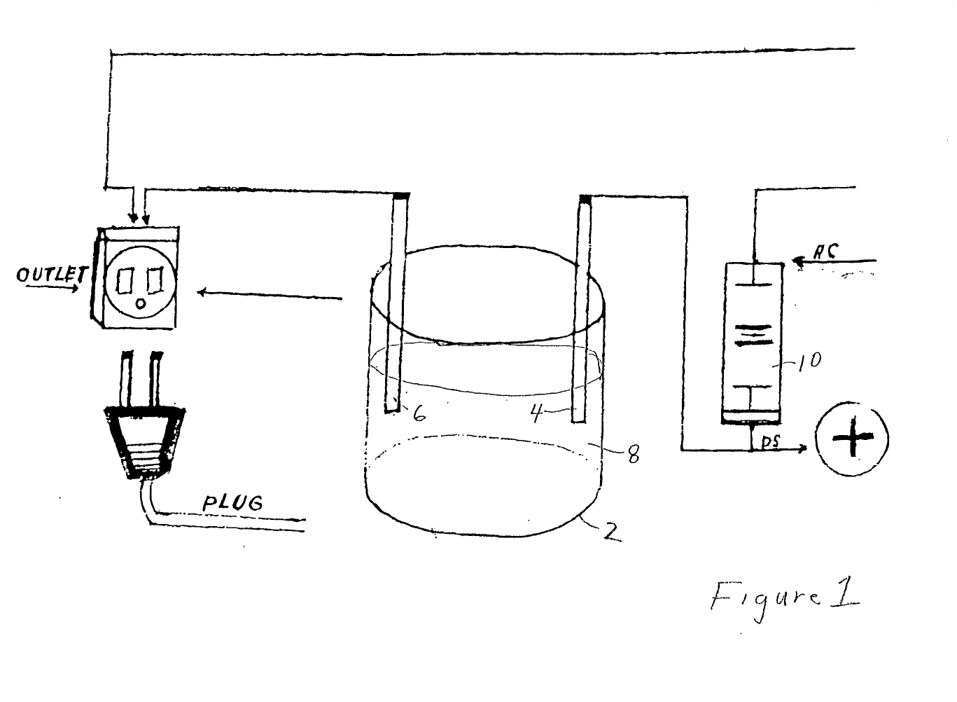

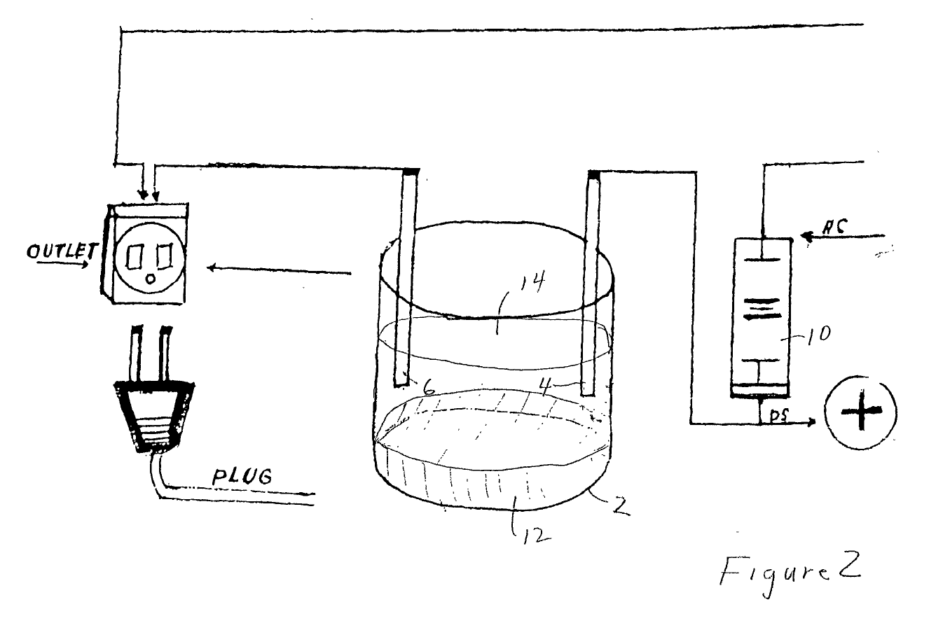

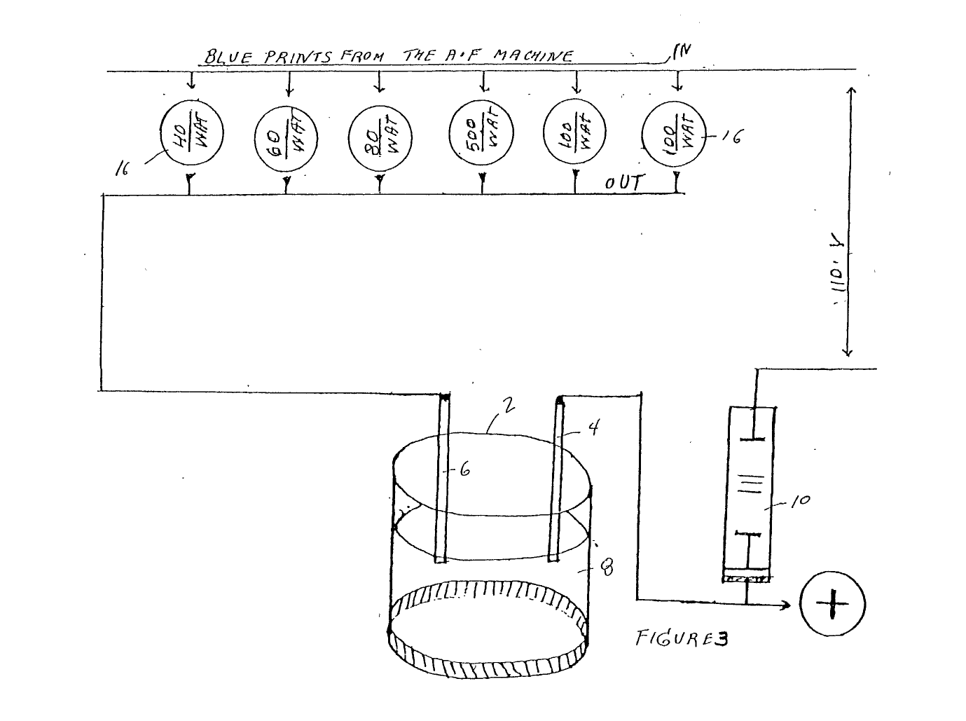

### DETAILED DESCRIPTION OF THE PREFERRED EMBODIMENT

Jalapeno pepper juice is obtained. The jalapeno pepper juice may be obtained by extracting the juice from jalapeno peppers by known means. For example, a food processor or similar device may be used to grind the jalapeno pepper, with the ground jalapeno pepper subsequently squeezed to extract the juice. The juice is then strained to obtain the juice.

The jalapeno pepper juice is then placed into a beaker 2. The beaker is preferred to be formed of a non-conductive material, such as glass. A metallic electrode is inserted into the beaker. In the preferred embodiment, the electrode electrode 4 is copper. A second, electrically conductive electrode 6 is also placed into the beaker. The second electrode may comprise copper. The electrodes are positioned to contact the jalapeno pepper juice 8, but do not contact each other.

The electrodes are connected to a current source, such as a direct current source. The direct current may be transformed from standard household current, which, in the United States, is 110 volts alternating current by means of a transformer. Two hundred (200) watts of AC power may be provided to the transformer. Higher, or lower, voltages and wattages may be used. As shown in the drawing figures, a semiconductor 10 may be used as a half wave rectifier to which household current is used to supply a current to the electrode 4. A silicon controlled rectifier could be used.

The current is provided to the jalapeno pepper juice though electrode 4. Current is supplied for the desired period of time. Typically, current is supplied until the desired quantity of the medicinal powder is present in the bottom of the beaker. The powder comprises copper, which is produced from the electrode, and the jalapeno pepper product. It should be understood that the resulting powder comprises both and copper and non-liquid jalapeno pepper, and that the components are not separate and distinguishable to the naked eye. The process as described produces a powder 12 comprising approximately 50% copper and 50% jalapeno pepper, when approximately ⅓ to ½ of the content in the beaker is powder and the remainder of the content is a supernatant liquid having a bluish green tint.

By empirical observation, four ounces of powder can be obtained by applying 200 watts of power for approximately one hour. An increase in power will reduce the time required to produce the same weight of powder

Referring to FIG. 3, a set of light bulbs 16 are in a parallel circuit. The light bulbs are preferred to be of varying wattages, as shown. The light bulbs are screwed in, or unscrewed, as desired, and are thereby to introduced into the circuit to vary the voltage across the electrodes as desired. In practice, light bulbs are introduced until the light bulbs are glowing with slightly less brightness than if they were in a light fixture and not in the present circuit. When this level of brightness is achieved, the voltage across the electrodes is deemed to be correct for the volume of juice to be processed. A rheostat or voltage divider could be used in place of the light bulbs.

The electrode that is connected to the rectifier is copper and contributes copper to the production of the powder. The second electrode conducts electricity. It may, but need not, comprise copper.

The resulting powder composition is topically applied as a medicine. It may be combined with an inert vehicle for topical application. The vehicle may be a cream, emollient, lotion or salve, such as petroleum jelly.

Other fruit or vegetable products may be used alone or in combination, although jalapeno pepper juice has been demonstrated to achieve particularly good results.

The device depicted in the drawing figures may be used to refine precious metals. An alloyed electrode comprising a precious metal, such as gold alloy, may be introduced to a charge from the transformer or rectifier. The electrode is present in a beaker comprising salt water, and opposite another electrode as shown in the drawing figures. Current is applied. After the electrode is deposited into the salt water by the current, the current is terminated, and nitric acid is introduced into the beaker. Water is then added. The alloy is poured off with the water, and pure gold remains in the beaker. The process as described will separate the gold from the alloy. Other precious metals may be similarly refined.

What is claimed is:

1. A medicine for topical application produced by the process of:
a. obtaining juice from a plant;
b. placing said juice in a non-electrically conductive container;
c. introducing a first electrically conductive and metallic electrode and a second electrically conductive electrode into said juice in said container;
d. applying a current to said second electrode and said first electrode;
e. continuing to apply said current until a desired amount of material is produced that comprises components from said plant and from said first electrically conductive and metallic electrode;
wherein said medicine is produced from said material.

2. A medicine for topical application produced by the process of:
a. obtaining juice from a plant;
b. placing said juice in a non-electrically conductive container;
c. introducing a first electrically conductive and metallic electrode and a second electrically conductive electrode into said juice in said container;
d. applying negatively charged direct current to said second electrode and a positively charged direct current to said first electrode;
e. continuing to apply said current until a desired amount of material is produced that comprises components from said plant and said first electrically conductive and metallic electrode;
wherein said medicine is produced from said material.

3. A medicine for topical application produced by the process of claim 1, wherein said first electrically conductive and metallic electrode comprises copper.

4. A medicine for topical application produced by the process of claim 2, wherein said first electrically conductive and metallic electrode comprises copper.

5. A medicine for topical application produced by the process of claim 1, wherein said juice is obtained from jalapeno pepper..

6. A medicine for topical application produced by the process of claim 2, wherein said juice is obtained from jalapeno pepper.

7. A medicine for topical application produced by the process of claim 1, wherein said juice is obtained from jalapeno pepper.

8. A medicine for topical application produced by the process of claim 2, wherein said juice is obtained from jalapeno pepper.
